(19) United States
(12) Patent Application Publication
(10) Pub. No.: US 2025/0073242 A1
(43) Pub. Date: Mar. 6, 2025

(54) SLC26A3 INHIBITORS AND USE THEREOF

(71) Applicant: THE REGENTS OF THE UNIVERSITY OF CALIFORNIA, Oakland, CA (US)

(72) Inventors: Alan S. VERKMAN, Oakland, CA (US); Onur CIL, Oakland, CA (US); Peter M. HAGGIE, Oakland, CA (US)

(21) Appl. No.: 18/292,329

(22) PCT Filed: Jul. 29, 2022
(86) PCT No.: PCT/US2022/038905

§ 371 Date: Jan. 25, 2024

Related U.S. Application Data
(60) Provisional application No. 63/227,926, filed on Jul. 30, 2021.

(51) Int. Cl. A61K 31/536; A61K 31/343; A61K 31/4035; A61K 31/433; A61K 31/519; A61P 13/02

(52) U.S. Cl. CPC A61K 31/536; A61K 31/343; A61K 31/4035; A61K 31/433; A61K 31/519; A61P 13/02

(57) ABSTRACT

Provided herein are inhibitors of SLC26A3, which is an anion (Cl⁻, HCO₃−, oxalate) exchanger expressed in intestinal epithelial cells. SLC26A3 inhibitors have potential utility for treatment of constipation including chronic idiopathic constipation (CIC), opioid-induced constipation (OIC), constipation-predominant irritable bowel syndrome (IBS-C), cystic fibrosis-associated constipation, meconium ileus, distal intestinal obstruction syndrome, calcium oxalate kidney stone disease, enteric hyperoxaluria and primary hyperoxalurias.

### STATEMENT OF GOVERNMENT INTEREST

This invention was made with government support under Grant Nos. DK 126070 and DK072517 awarded by The National Institutes of Health. The government has certain rights in the invention.

### BACKGROUND

### Technical Field

This disclosure is related to selective inhibitors of SLC26A3, an anion exchanger in gastrointestinal tract and use thereof.

Description of the Related Art

SLC26A3, originally named DRA (down-regulated in adenoma), is an anion (Cl⁻, HCO₃⁻, oxalate) exchanger expressed in the luminal membrane of intestinal epithelial cells. DRA loss of function in humans or mice causes chloride-losing diarrhea. SLC26A3 is also the main transporter in the gut for facilitating the absorption of oxalate. DRA knock-out mice have 60% lower serum oxalate levels and 70% lower urine oxalate levels.

WO 2019/210103 discloses several classes of small-molecule SLC26A3 inhibitors, which were shown to be effective in inhibiting intestinal fluid absorption and oxalate absorption. In particular, inhibition of intestinal fluid absorption was demonstrated in closed intestinal loops in mice, and efficacy was demonstrated in an experimental model of constipation. Moreover, SLC26A3 inhibition and NHE3 inhibition appear to provide an additive or synergistic effect, which can be highly effective in treating refractory constipation.

Furthermore, certain small molecule SLC26A3 inhibitors were also demonstrated to be capable of preventing or treating hyperoxaluria and renal failure by decreasing the amount of oxalate excreted in urine, which is achieved by inhibiting the intestinal absorption of oxalate and removing the unabsorbed oxalate through stool, instead of urine. See also WO 2019/210103.

There remains a need in the art for improved therapy for treating constipation, hyperoxaluria and kidney stones by targeting SLC26A3.

### BRIEF SUMMARY

Provided herein are five classes (E, F, G, H and I) of potent inhibitors of SLC26A3. In particular, the SLC26A3 inhibitors disclosed herein are demonstrated to act from outside of the intestinal epithelial cells, making them luminally-acting agents that can be developed into drugs with minimal systemic availability.

By selectively targeting SLC26A3, compounds and compositions disclosed herein are shown to be effective in inhibiting intestinal fluid absorption, thus providing therapy for treating constipation.

Moreover, the present disclosure demonstrates a direct link between SLC26A3 inhibition and a reduced intestinal oxalate absorption. SLC26A3 inhibition is an effective therapy for preventing or treating hyperoxaluria, calcium oxalate nephrolithiasis and renal failure by decreasing the amount of oxalate excreted in urine, which is achieved by inhibiting the intestinal absorption of oxalate and removing the unabsorbed oxalate through stool, instead of urine.

These and other aspects of the disclosure will be apparent upon reference to the detailed description below.

### BRIEF DESCRIPTION OF THE SEVERAL VIEWS OF THE DRAWINGS

FIG. 1 shows the concentration-dependence of SLC26A3 inhibition for Class E compounds identified in screening.

FIG. 2 shows the concentration-dependence of SLC26A3 inhibition for Class F compounds identified in screening.

FIG. 3 shows the concentration-dependence of SLC26A3 inhibition for Class G compounds identified in screening.

FIG. 4 shows the concentration-dependence of SLC26A3 inhibition for Class H compounds identified in screening.

FIG. 5 shows the concentration-dependence of SLC26A3 inhibition for Class I compounds identified in screening.

FIG. 6 demonstrates the extracellular site of action of a G class compound and a H class compound.

FIG. 7 shows the effect of a known SLC26A3 inhibitor (DRAᵢₙₕ-A270) on oxalate transport at physiologically relevant concentrations.

FIG. 8A and FIG. 8B show DRAᵢₙₕ-A270 prevents intestinal oxalate absorption and reduces hyperoxaluria.

### DETAILED DESCRIPTION

SLC26A3 is highly expressed in the gastrointestinal (GI) tract and known to mediate anion exchange. Targeting SLC26A3 is demonstrated herein as an effective therapy for treating or preventing constipation by inhibiting fluid absorption. Similarly, targeting SLC26A3 is shown to be effective in preventing hyperoxaluria, kidney stone diseases and oxalate nephropathy by inhibiting intestinal oxalate absorption. See e.g., WO 2019/210103, the content of which is incorporated herein by reference in its entirety.

Five classes of potent SLC26A3 inhibitors are identified by high throughput screening of SLC26A3 inhibition and follow-on structure-activity studies. These inhibitors are suitable for preventing or treating a condition, disease, or disorder associated with SLC26A3-mediated anion exchange. In particular, some of the compounds of the present disclosure are demonstrated to act from outside of cells, thus allowing the development of membrane-impermeable and non-absorbable SLC26A3 inhibitors. By limiting the site of action locally within the GI tract, the SLC26A3 inhibitors can have minimal systemic availability and be eliminated in the stool.

Thus, one embodiment provides an anti-absorptive therapy for constipation by administering to a subject in need thereof one or more SLC26A3 inhibitors disclosed herein. It is believed that inhibition of SLC26A3, alone or together with drugs acting on alternative anti-absorptive or pro-secretory mechanisms, could be highly effective in treating refractory constipation, including chronic idiopathic constipation (CIC), irritable bowel syndrome with constipation (IBS-C), opioid-induced constipation (OIC) and cystic fibrosis (CF)-associated constipation, meconium ileus and distal intestinal obstruction syndrome. Importantly, the compounds of the present disclosure exhibited selectivity for slc26a3 and did not inhibit homologous slc26a-family anion exchangers or relevant intestinal transporters.

In another embodiment, inhibiting SLC26A3 decreases oxalate absorption, thereby protecting kidneys from detrimental effects of hyperoxaluria seen in calcium oxalate kidney stone disease, enteric hyperoxaluria and primary hyperoxaluria. The SLC26A3 inhibitors suitable for therapies for constipation or hyperoxaluria are described in further detail below.

### Class E Compounds

One embodiment provides a compound of Formula (I) for preventing or treating a condition, disease, or disorder associated with SLC26A3-mediated anion exchange:

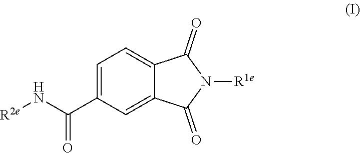

wherein,

R^{1e} is (i) phenyl optionally substituted with one or more substituents selected from the group consisting of C₁₋₆alkyl, halo, −NO₂, phenyl, and C₁₋₆alkoxy; (ii) C₃₋₆ alkyl, or (iii) heterocyclylC₁₋₄alkyl; and

R^{2e} is carboxy-substituted phenyl, optionally further substituted with one or more substituents selected from the group consisting of C₁₋₆alkyl, halo, hydroxyl and C₁₋₆alkoxy.

In more specific embodiments, R^{1e} is substituted phenyl, including without limitation, 4-chlorophenyl, 3,4-dimethylphenyl, 4-methoxyphenyl, 1,1′-biphenyl, 2-methyl-4-nitrophenyl, 2,4,6-trimethylphenyl, or 3-nitrophenyl.

In more specific embodiments, R^{1e} is C₃₋₆ alkyl, including without limitation, propyl, 2-methylpropyl, or 3-methylbutyl.

In other more specific embodiments, R^{1e} is

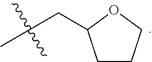 [SMILES: *CC1CCCO1]

A more specific embodiment provides a compound of Formula (Ia):

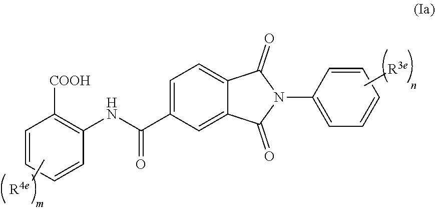

wherein,

m is 0, 1 or 2;

n is 1, 2 or 3;

R^{3e} is C₁₋₆alkyl, halo, —NO₂, phenyl, and C₁₋₆alkoxy;

R^{4e} is C₁₋₆-alkyl, halo, hydroxyl or C₁₋₆alkoxy.

In preferred embodiments, R^{3e} is methyl, chloro, —NO₂, phenyl, or methoxy.

In preferred embodiments, m is 0.

In other embodiments, n is 1 or 2; and R^{4e} is 4-hydroxy, 5-chloro, 4,5-dimethoxy, 4-methyl or 4-bromo.

The IC₅₀ data of certain compounds of Formula (I) are shown in Table 1.

TABLE 1

| Compound | | | |
|---|---|---|---|
| (DRAᵢₙₕ-Exxxx) | R^{1e} | R^{2e} | IC₅₀ (nM) |
| 0324 | 4-Cl-Phenyl | 2-carboxy-phenyl | 210 |
| 2333 | 3,4-dimethyl-Phenyl | 2-carboxy-phenyl | 310 |
| 0078 | 1-Isobutyl | 2-carboxy-phenyl | 690 |
| 2216 | 4-OMe-phenyl | 2-carboxy-phenyl | >90% at 2500 |
| 0329 | Butyl | 2-carboxy-phenyl | >90% at 5000 |
| 9590 | 1,1′-Biphenyl | 2-carboxy-phenyl | >90% at 5000 |
| 3073 | 2-Me-4-NO₂-phenyl | 2-carboxy-phenyl | >90% at 5000 |
| 0982 | 2- | 2-carboxy-phenyl | >90% at 5000 |
| | Methyltetrahydrofuran | | |
| 1900 | 1,1′-Biphenyl | 2-carboxy-5-Cl- | >90% at 5000 |
| | | phenyl | |
| 1422 | 2,4,6-trimethyl-phenyl | 2-carboxy-4,5- | >90% at 25000 |
| | | OMe-phenyl | |
| 2922 | 3-NO₂-phenyl | 2-carboxy-phenyl | >90% at 25000 |
| 2951 | 2,4,6-trimethyl-phenyl | 2-carboxy-phenyl | >90% at 25000 |
| 1587 | 1-Isopropyl | 2-carboxy-4- | >90% at 25000 |
| | | hydroxy-phenyl | |

In preferred embodiments, the compound of Formula (I) has one of the following structures:

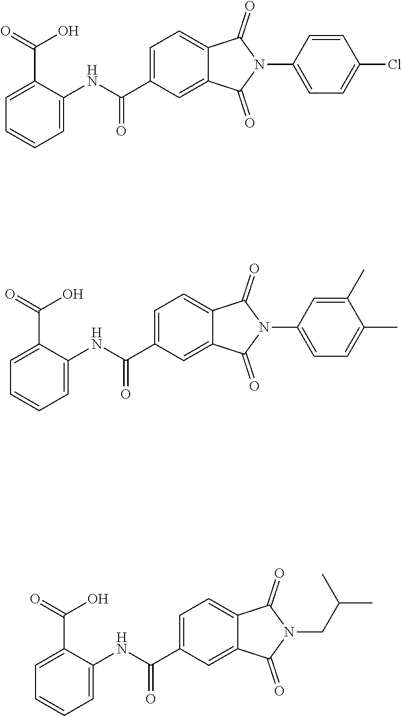 [SMILES: O=C(Nc1ccccc1C(=O)O)c1ccc2c(c1)C(=O)N(c1ccc(Cl)cc1)C2=O ; Cc1ccc(N2C(=O)c3ccc(C(=O)Nc4ccccc4C(=O)O)cc3C2=O)cc1C ; CC(C)CN1C(=O)c2ccc(C(=O)Nc3ccccc3C(=O)O)cc2C1=O]

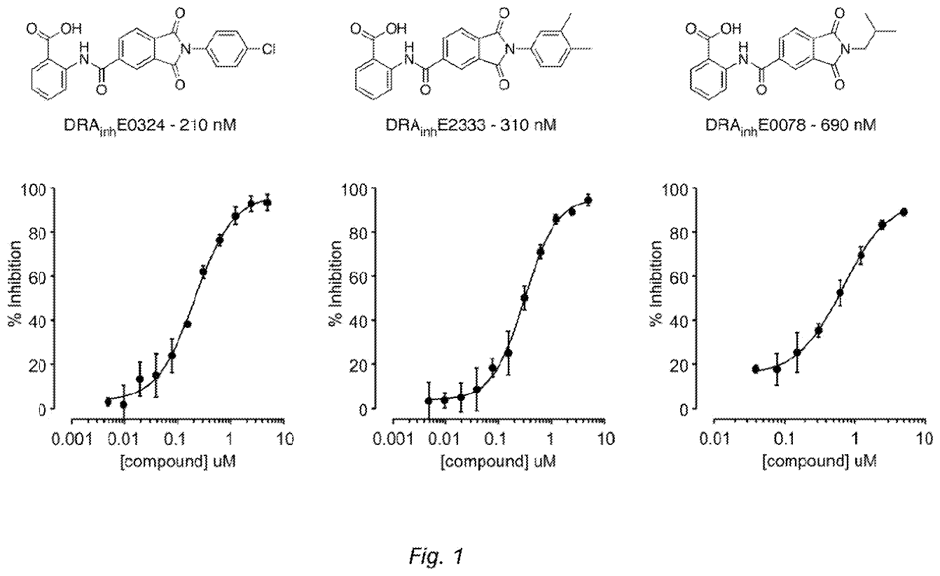

FIG. 1 shows concentration-dependence of SLC26A inhibition for the above three representative compounds of Formula (I) using fitted curves for a single-site inhibition model.

### Class F Compounds

One embodiment provides a compound of Formula (II) for preventing or treating a condition, disease, or disorder associated with SLC26A3-mediated anion exchange:

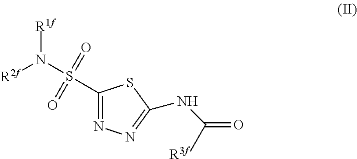

wherein,

R^{1f} is hydrogen or C₁₋₃alkyl;

R^{2f} is phenyl optionally substituted with one or more substituents selected from the group consisting of C₁₋₆alkyl, halo, and C₁₋₆alkoxy; or

R^{1f} and R^{2f} together with the nitrogen to which they are connected form a heteroaryl optionally substituted with C₁₋₃alkyl; and

R^{3f} is

(i) phenyl optionally substituted with one or more substituents selected from the group consisting of C₁₋₆alkyl, halo, —NO₂, and C₁₋₆alkoxy;

(ii) C₅₋₆ cycloalkyl or C₅₋₆ heteroaryl;

(iii)

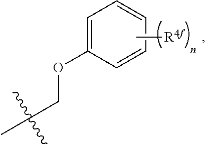

wherein n is 0, 1 or 2, R^{4f} is C₁₋₆alkyl, halo, or C₁₋₆alkoxy.

In preferred embodiments, R^{1f} is hydrogen. In other embodiments, R^{1f} is methyl or ethyl.

In more specific embodiments, R^{2f} is phenyl optionally substituted with one or more substituents selected from the group consisting of methyl, chloro, and methoxy. In even more specific embodiments, R^{2f} is 3-methylphenyl, 2-methylphenyl, 4-methoxyphenyl, 3,5-dimethylphenyl, phenyl, 4-methylphenyl, 4-methoxyphenyl, 4-chlorophenyl, or 2,4-dimethylphenyl.

In other embodiments, R^{1f} and R^{2f} together with the nitrogen to which they are connected form:

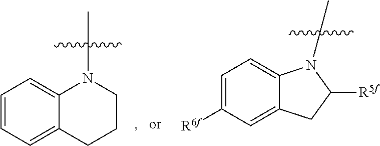

wherein R^{5f} and R^{6f} are independently hydrogen or C₁₋₃alkyl (e.g., methyl).

In more specific embodiments, R^{3f} is phenyl optionally substituted with one or more substituents selected from the group consisting of methyl, fluoro, chloro, bromo, —NO₂, methoxy, and ethoxy.

In even more specific embodiments, R^{3f} is 3-methylphenyl, 3-methoxyphenyl, 2-fluorophenyl, 4-methylphenyl, 4-chlorophenyl, 4-bromophenyl, 3,4-dimethylphenyl, 3,4-dimethoxyphenyl, 3,4,5-trimethoxyphenyl, phenyl, 2-methoxyphenyl, 2-fluorophenyl, 3-fluorophenyl, 3-chlorophenyl, 4-fluorophenyl, 3-methoxyphenyl, 4-methoxyphenyl, 3-chlorophenyl, 3-bromophenyl, 3-nitrophenyl, or 3,4,5-triethoxyphenyl.

In more specific embodiments, R^{3f} is thienyl or furanyl.

In more specific embodiments, R^{3f} is

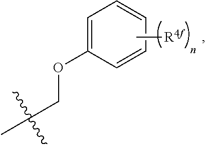

wherein n is 0, 1 or 2, R^{4f} is methyl, fluoro, chloro, and methoxy.

In even more specific embodiments, R^{4f} is 3,4-dimethylphenyl, 2-methylphenyl, 3-methylphenyl, 4-methylphenyl, 4-methoxyphenyl, 4-fluorophenyl, or 4-chlorophenyl.

The IC₅₀ data of certain compounds of Formula (II) are shown in Table 2.

TABLE 2

| Compound | | | | |
|---|---|---|---|---|
| (DRAᵢₙₕ-Fxxxx) | R^{1f} | R^{2f} | R^{3f} | IC₅₀ (nM) |
| 6984 | Methyl | 3,5-Me-phenyl | 3,4-Me- | 260 |
| | | | methoxyphenyl | |
| 0165 | H | 3-Me-phenyl | 3-Me-phenyl | 470 |
| 0170 | H | 3-Me-phenyl | 3-OMe-phenyl | 310 |
| 2644 | Indoline | | 4-Me-methoxyphenyl | 2600 |
| 0096 | H | 2-Me-phenyl | 3,4-OMe-phenyl | >90% at 5000 |
| 0103 | H | 2-Me-phenyl | 3,4-Me-phenyl | >90% at 5000 |
| 0156 | H | 3-Me-phenyl | 4-Cl-phenyl | >90% at 5000 |
| 0172 | H | 3-Me-phenyl | 3,4-OMe-phenyl | >90% at 5000 |
| 0179 | H | 3-Me-phenyl | 3,4-Me-phenyl | >90% at 5000 |
| 2594 | Indoline | | 3-Br-phenyl | >90% at 5000 |
| 2641 | Indoline | | 3-Me-phenyl | >90% at 5000 |
| 2642 | Indoline | | 4-Me-phenyl | >90% at 5000 |
| 2646 | Indoline | | 4-F-phenyl | >90% at 5000 |
| 2665 | 3,4-Dihydroquinolin-1(2H)-yl | | 3-NO₂-phenyl | >90% at 5000 |
| 2683 | 3,4-Dihydroquinolin-1(2H)-yl | | 3-Cl-phenyl | >90% at 5000 |
| 2718 | 3,4-Dihydroquinolin-1(2H)-yl | | 4-Me-methoxyphenyl | >90% at 5000 |
| 2719 | 3,4-Dihydroquinolin-1(2H)-yl | | 3,4-Me- | >90% at 5000 |
| | | | methoxyphenyl | |
| 5483 | Methyl | Phenyl | 3,4-Me- | >90% at 5000 |
| | | | methoxyphenyl | |
| 5698 | Ethyl | 2-Me-phenyl | 4-F-phenyl | >90% at 5000 |
| 5699 | Ethyl | 2-Me-phenyl | 4-Cl-phenyl | >90% at 5000 |
| 5702 | Ethyl | 2-Me-phenyl | 4-Me-phenyl | >90% at 5000 |
| 5715 | Ethyl | 2-Me-phenyl | 3-Me-phenyl | >90% at 5000 |
| 5756 | Methyl | 2-Me-phenyl | 4-F-phenyl | >90% at 5000 |
| 5760 | Methyl | 2-Me-phenyl | 4-Me-phenyl | >90% at 5000 |
| 5765 | Methyl | 2-Me-phenyl | 4-OMe-phenyl | >90% at 5000 |
| 5773 | Methyl | 2-Me-phenyl | 3-Cl-phenyl | >90% at 5000 |
| 6334 | Ethyl | 4-Me-phenyl | 4-F-phenyl | >90% at 5000 |
| 6392 | Methyl | 4-Me-phenyl | 4-F-phenyl | >90% at 5000 |
| 6412 | Methyl | 4-Me-phenyl | 3-OMe-phenyl | >90% at 5000 |
| 6433 | Methyl | 4-Me-phenyl | Methoxyphenyl | >90% at 5000 |
| 6900 | Ethyl | 3,5-Me-phenyl | 3-F-phenyl | >90% at 5000 |
| 7156 | Ethyl | 2,4-Me-phenyl | Phenyl | >90% at 5000 |
| 7169 | Ethyl | 2,4-Me-phenyl | 3-OMe-Phenyl | >90% at 5000 |
| 7171 | Ethyl | 2,4-Me-phenyl | 3,4-OMe-Phenyl | >90% at 5000 |
| 8358 | Ethyl | 4-Cl-phenyl | 4-Me-phenyl | >90% at 5000 |
| 8360 | Ethyl | 4-Cl-phenyl | 2-Me-phenyl | >90% at 5000 |
| 8368 | Ethyl | 4-Cl-phenyl | 3,4-OMe-phenyl | >90% at 5000 |
| 8413 | Methyl | 4-Cl-phenyl | 4-Me-phenyl | >90% at 5000 |
| 8415 | Methyl | 4-Cl-phenyl | 2-Me-phenyl | >90% at 5000 |
| 8416 | Methyl | 4-Cl-phenyl | 3-Me-phenyl | >90% at 5000 |

In preferred embodiments, the compound of Formula (II) has one of the following structures:

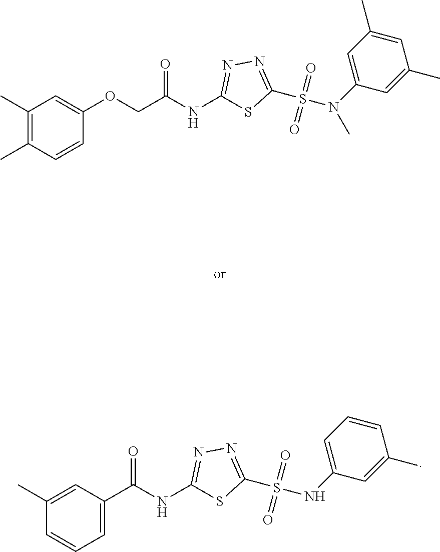

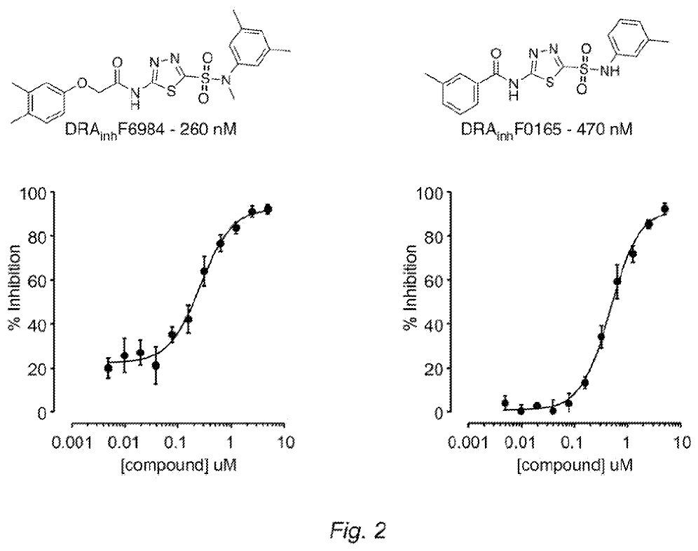

FIG. 2 shows concentration-dependence of SLC26A3 inhibition for the above two representative compounds of Formula (II) using fitted curves for a single-site inhibition model.

### Class G Compounds

One embodiment provides a compound of Formula (III) for preventing or treating a condition, disease, or disorder associated with SLC26A3-mediated anion exchange:

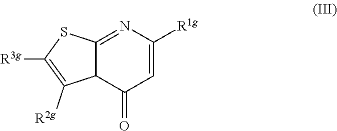

wherein,

R^{1g} is

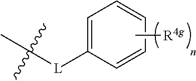

wherein, n is 0, 1, 2, or 3;

L is —(CH₂)ₘ—X—(CH₂)ₚ—, wherein m and p are independently 0 or 1, X is —O—, —S—, —N(R^{5g})—, or —OC(O)—;

R^{2g} is phenyl optionally substituted with one or more substituents selected from the group consisting of C₁₋₄alkyl and C₁₋₄alkoxy;

R^{3g} is hydrogen or C₁₋₄alkyl;

R^{4g} is C₁₋₄alkyl, halo, or C₁₋₄alkoxy; or two adjacent R^{4g} and the carbons to which they are attached form a 5-member or 6-member heteroaryl; and

R^{5g} is hydrogen or C₁₋₃alkyl.

In more specific embodiments, R^{2g} is phenyl or 4-methoxyphenyl, R^{3g} is hydrogen, and R^{1g} has one of the following structures:

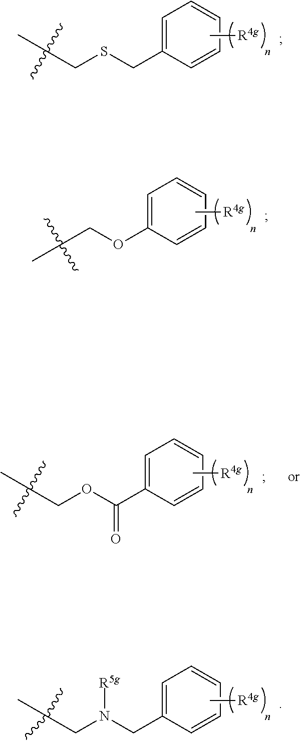

In more specific embodiments, n is 0.

In other specific embodiments, n is 1, 2 or 3, and R^{4g} is chloro, methyl, fluoro, methoxy, or two adjacent R⁴⁸ and the carbons to which they are attached form 3,4-benzodioxol.

In even more specific embodiments, when R^{1g} is:

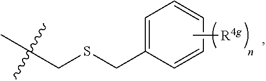

n is 0, or nis 1, and R^{4g} is 4-chloro.

In other more specific embodiments, when R^{1g} is:

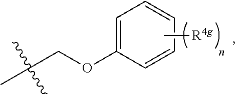

n is 1, 2 or 3; and R^{4g} is 2-chloro, 2,4-dichloro, 2,4-dimenthyl, 3-methyl, or 2,5-dichloro.

In other more specific embodiments, when R^{1g} is:

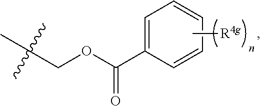

n is 0, or n is 1, and R^{4g} is 2-methyl, 3-methyl, 4-fluoro, 4-chloro, 4-methyl, 4-methoxy, or two adjacent R^{4g} and the carbons to which they are attached form 3,4-benzodioxol.

In other more specific embodiments, when R^{1g} is:

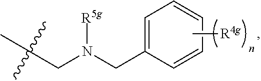

R^{5g} is methyl or ethyl, and n is 0, or n is 1, and R^{4g} is 4-chloro, 4-methyl, or 4-fluoro.

The IC₅₀ data of certain compounds of Formula (III) are shown in Table 3.

TABLE 3

| Compound | | | | |
|---|---|---|---|---|
| (DRAᵢₙₕ- | | | | |
| Gxxxx) | R^{1g} | R^{2g} | R^{3g} | IC₅₀ (nM) |
| 2420 | 2-Cl-methoyxphenyl | Phenyl | H | 360 |
| 2253 | (4-Cl-benzyl)thiomethyl | Phenyl | H | 540 |
| 3858 | 2,4-Cl-methoyxphenyl | Phenyl | H | 1300 |
| 0813 | 4-Methyl-methylbenzoate | Phenyl | H | 2300 |
| 2416 | 2,4-Me-methoyxphenyl | Phenyl | H | >90% at 5000 |
| 0826 | 3-Methyl-methylbenzoate | Phenyl | H | >90% at 5000 |
| 0852 | 3,4-Dioxole- methylbenzoate | Phenyl | H | >90% at 5000 |
| 0144 | (Benzyl(ethyl)amino)methyl | 4-OMe- | H | >90% at 5000 |
| | | phenyl | | |
| 0296 | 4-F-(Benzyl(ethyl)amino)methyl | Phenyl | H | >90% at 5000 |
| 0374 | 4-Cl-(Benzyl(ethyl)amino)methyl | Phenyl | H | >90% at 5000 |
| 0378 | 4-Cl-(Benzyl(ethyl)amino)methyl | 4-OMe- | H | >90% at 5000 |
| | | phenyl | | |

In preferred embodiments, the compound of Formula (III) has one of the following structures:

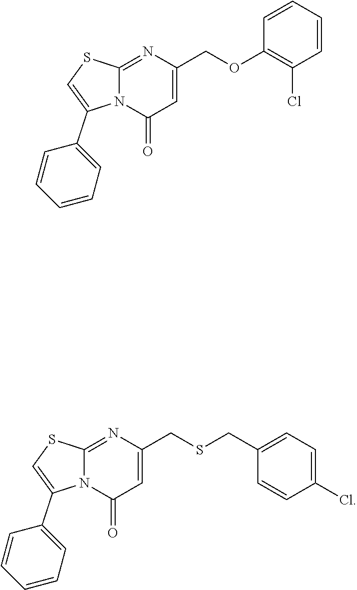 [SMILES: O=c1cc(COc2ccccc2Cl)nc2scc(-c3ccccc3)n12 ; O=c1cc(CSCc2ccc(Cl)cc2)nc2scc(-c3ccccc3)n12]

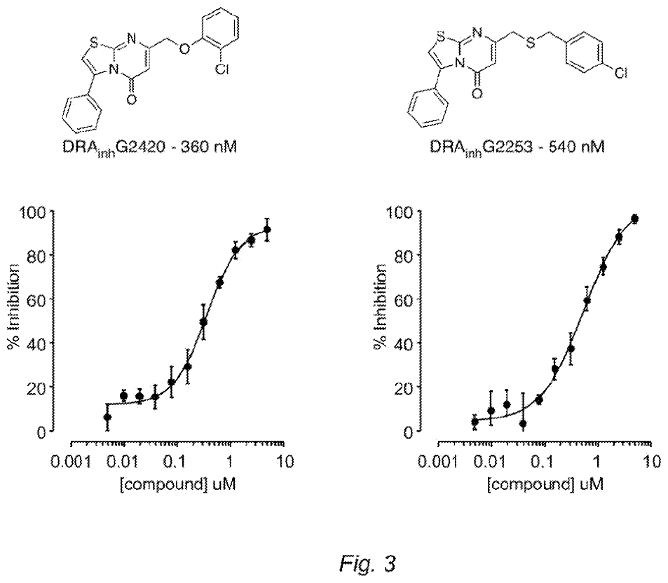

FIG. 3 shows concentration-dependence of SLC26A3 inhibition for the above two representative compounds of Formula (III) using fitted curves for a single-site inhibition model.

### Class H Compounds

One embodiment provides a compound of Formula (IV) for preventing or treating a condition, disease, or disorder associated with SLC26A3-mediated anion exchange:

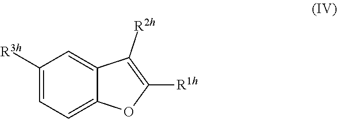

wherein,

R^{1h} is C₁₋₃alkyl or phenyl optionally substituted with one or more substituents selected from the group consisting of C₁₋₄alkyl, halo, and C₁₋₄alkoxy;

R^{2h} is —COOR^{5h};

R^{3h} is

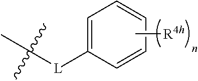

wherein, n is 0, 1, or 2;

L is —X—(CH₂)ₘ—Y—(CH₂)ₚ—, wherein m and p are independently 0 or 1, X is —O— or —S—, Y is a direct bond or —C(O)O—;

R^{4h} is C₁₋₄alkyl or halo; and

R^{4h} is hydrogen or C₁₋₄alkyl.

In more specific embodiments, R^{1h} is phenyl or methyl, R^{2h} is —COOH, and R^{3h} has the following structure:

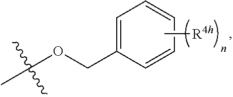

wherein n is 1 or 2, and R^{4h} is methyl, fluoro, chloro, or bromo.

In more specific embodiments, R^{4h} is 4-methyl, 2-methyl, 2-chloro, 3-chloro, 4-chloro, 2-chloro-6-fluoro, 4-bromo, 3,4-dichloro, 2,4-dichloro.

In other more specific embodiments, n is 0.

In further embodiments, R^{1h} is methyl, R^{2h} is-COOCH₃, and R^{3h} has the following structure:

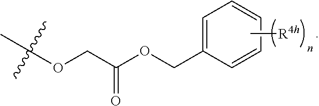

In preferred embodiments, n is 0.

The IC₅₀ data of certain compounds of Formula (IV) are shown in Table 4.

TABLE 4

| Compound | | | | |
|---|---|---|---|---|
| (DRAᵢₙₕ- | | | | IC₅₀ |
| Hxxxx) | R^{1h} | R^{2h} | R^{3h} | (nM) |
| 0163 | Phenyl | Carboxylate | (4-Methyl-benzyl)oxy- | 190 |
| 0696 | Phenyl | Carboxylate | (2-Methyl-benzyl)oxy- | 340 |
| 0365 | Phenyl | Carboxylate | (2-Cl-benzyl)oxy- | 690 |
| 0657 | Phenyl | Carboxylate | (3-Cl-benzyl)oxy- | 1200 |
| 0354 | Phenyl | Carboxylate | (4-Cl-benzyl)oxy- | 1700 |
| 0493 | Phenyl | Carboxylate | (3,4-Cl-benzyl)oxy- | 2300 |
| 0546 | Phenyl | Carboxylate | (Benzyl)oxy- | 2600 |
| 0368 | Methyl | Methyl-carboxylate | Benzyl-2-methoxyacetate | 9800 |

In preferred embodiments, the compound of Formula (IV) has one of the following structures:

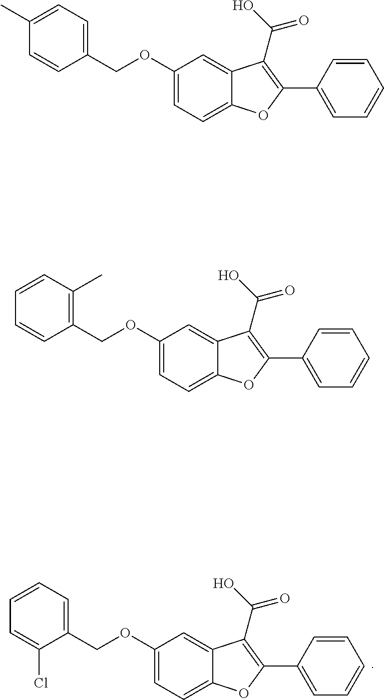 [SMILES: Cc1ccc(COc2ccc3oc(-c4ccccc4)c(C(=O)O)c3c2)cc1 ; Cc1ccccc1COc1ccc2oc(-c3ccccc3)c(C(=O)O)c2c1 ; O=C(O)c1c(-c2ccccc2)oc2ccc(OCc3ccccc3Cl)cc12]

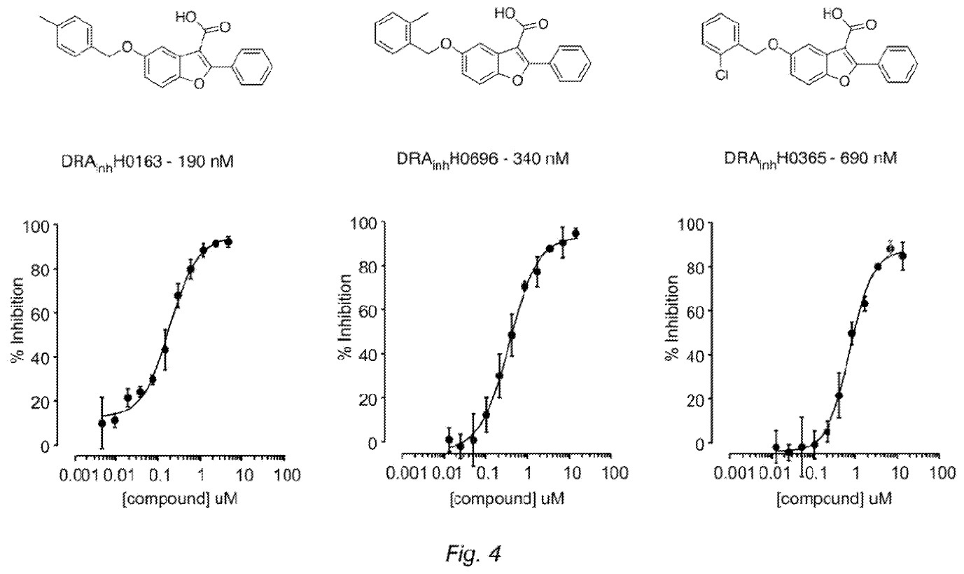

FIG. 4 shows concentration-dependence of SLC26A3 inhibition for the above three representative compounds of Formula (IV) using fitted curves for a single-site inhibition model.

### Class I Compounds

One embodiment provides a compound of Formula (V) for preventing or treating a condition, disease, or disorder associated with SLC26A3-mediated anion exchange:

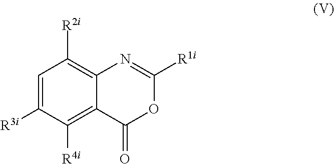

wherein,

R¹ⁱ is
(i) phenyl optionally substituted with one or more substituents selected from the group consisting of C₁₋₄alkyl, halo, —NO₂, C₁₋₄alkoxy and —NHC(O)—R⁵ⁱ; or
(ii)

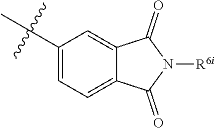

R²ⁱ is hydrogen or C₁₋₃alkyl;

R³ⁱ and R⁴ⁱ are hydrogen; and

R⁵ⁱ is
(i) C₁₋₄alkyl;
(ii) -L-phenyl, wherein phenyl is optionally substituted with one or more substituents selected from the group consisting of C₁₋₄alkyl, halo, C₁₋₄alkoxy and C(O)O—C₁₋₄alkyl; L is a direct bond or —(CH₂)ₘ—X—, and X is O or S;

R⁶ⁱ is C₁₋₄alkyl or phenyl optionally substituted with C₁₋₄alkyl.

In a more specific embodiment, L is direct bond, and the compound of Formula (V) has a structure represented by Formula (Va):

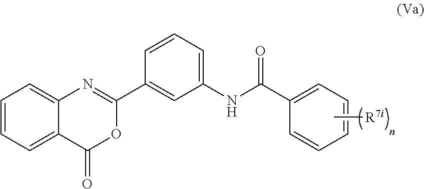

wherein,

n is 0, 1, or 2;

R⁷ⁱ is C₁₋₄alkyl, halo, C₁₋₄alkoxy or C(O)O—C₁₋₄alkyl.

In more specific embodiments, R⁷ⁱ is 4-chloro, 4-ethoxy, 2-bromo, 3-methyester, 4-iodo, or 4-methylester.

In other more specific embodiments, L is —(CH₂)—O—; and the compound of Formula (V) has a structure represented by Formula (Vb):

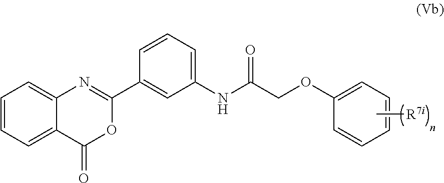

wherein,

n is 0, 1, or 2;

R⁷ⁱ is C₁₋₄alkyl, halo, C₁₋₄alkoxy or C(O)O—C₁₋₄alkyl.

In more specific embodiments, R⁷ⁱ is 2-methoxy, 2,6-dimethyl, 4-methoxy or 4-5 methyl.

In yet other more specific embodiments, Rli is phenyl substituted with 3-nitro, 3-chloro, 3-methylester, 4-methylester, 3-nitro-4-methyl, 3-methoxy-4-methylester, or 4-t-butyl.

In another specific embodiment, R¹ⁱ is phenyl substituted with —NHC(O)R⁵ⁱ, wherein R⁵ⁱ is methyl, isopropyl or t-butyl.

In further more specific embodiments, R¹ⁱ is

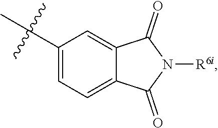

wherein R⁶ⁱ is

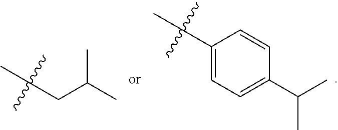

The IC₅₀ data of certain compounds of Formula (V) are shown in Table 5.

TABLE 5

| Compound | | | |
|---|---|---|---|
| (DRAᵢₙₕ- | | | |
| Ixxxx) | R¹ⁱ | R²ⁱ/R³ⁱ/R⁴ⁱ | IC₅₀ (nM) |
| 0066 | 2-Bromo-N-phenylbenzamide | H | 100 |
| 0541 | 4-Ethoxy-N-phenylbenzamide | H | 140 |
| 0081 | 4-Chloro-N-phenylbenzamide | H | 260 |
| 0757 | 2-(2-Methoxyphenoxy)-N- | H | 580 |
| | phenylacetamide | | |
| 0761 | 2-(2,6-Dimethylphenoxy)-N- | H | 740 |
| | phenylacetamide | | |
| 8955 | N-Phenylacetamide | H | 740 |
| 2364 | N-Phenylypivalamide | H | 750 |
| 0139 | N-Phenylisobutyramide | H | 980 |
| 0143 | 2-(4-Methylphenoxy)-N- | H | >90% at |
| | phenylacetamide | | 2500 |
| 0351 | 4-Methylacetate-N-phenylbenzamide | H | >90% at |
| | | | 5000 |
| 5672 | 4-Me-3NO₂-phenyl | H | >90% at |
| | | | 5000 |
| 6669 | 2-Isobutyl-5-isoindoline-1,3-dione | H | >90% at |
| | | | 5000 |
| 0106 | 3NO₂-phenyl | H | >90% at |
| | | | 5000 |
| 0451 | 2-(4-Methoxyphenoxy)-N- | H | >90% at |
| | phenylacetamide | | 5000 |
| 1811 | 4-Iodo-N-phenylbenzamide | H | >90% at |
| | | | 5000 |
| 1808 | 3-Bromo-N-phenylbenzamide | H | >90% at |
| | | | 5000 |

In preferred embodiments, the compound of Formula (V) has one of the following structures:

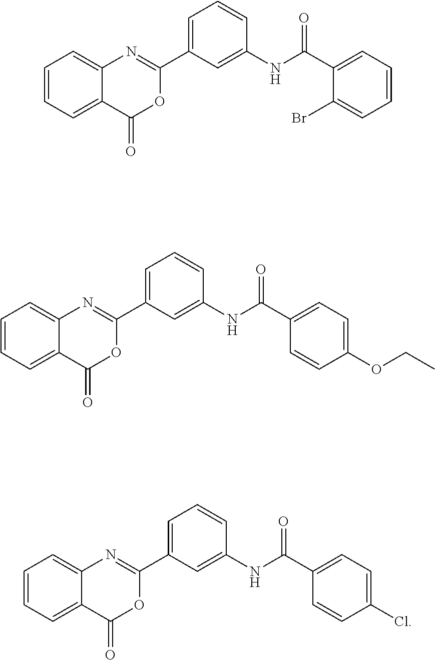 [SMILES: O=C(Nc1cccc(-c2nc3ccccc3c(=O)o2)c1)c1ccccc1Br ; CCOc1ccc(C(=O)Nc2cccc(-c3nc4ccccc4c(=O)o3)c2)cc1 ; O=C(Nc1cccc(-c2nc3ccccc3c(=O)o2)c1)c1ccc(Cl)cc1]

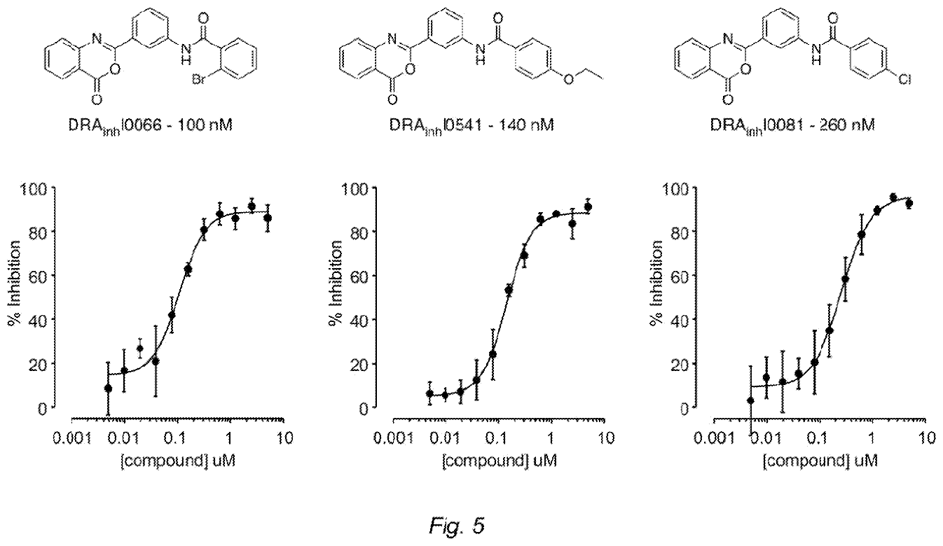

FIG. 5 shows concentration-dependence of SLC26A3 inhibition for three representative compounds of Formula (V) using fitted curves for a single-site inhibition model.

### Chemistry Definitions

“Alkyl” means a straight chain or branched, noncyclic, unsaturated or partially unsaturated aliphatic hydrocarbon containing from 1 to 12 carbon atoms. A lower alkyl refers to an alkyl that has any number of carbon atoms between 1 and 6 (i.e., C₁-C₆ alkyl) Representative saturated straight chain alkyls include methyl, ethyl, n-propyl, n-butyl, n-pentyl, n-hexyl, and the like, while saturated branched alkyls include isopropyl, sec-butyl, isobutyl, tert-butyl, tert-pentyl, heptyl, n-octyl, isopentyl, 2-ethylhexyl and the like. Alkyl may be optionally substituted by one or more substituents as defined herein.

“Alkoxy” refers to the radical of —O-alkyl. Examples of alkoxy include methoxy, ethoxy, and the like. The alkyl moiety of alkoxy may be optionally substituted by one or more substituents as defined herein.

“Alkoxyalkyl” refers to a radical of the formula —R_{b}ORₐ where Rₐ is an alkyl radical as defined above and R_{b} is an alkylene chain.

“Carboxyalkyl” refers to a straight or branched alkyl radical substituted with —CO₂H. The length of the alkyl radical may be indicated by the number of the carbon atoms excluding the carbon of the carboxy moiety, for example, carboxyC₁-C₃alkyl includes —CH₂CO₂H, —CH₂CH₂CO₂H, —CH₂CH₂CH₂CO₂H, and the like.

“Cycloalkyl” refers to a stable non-aromatic monocyclic or polycyclic carbocyclic radical consisting solely of carbon and hydrogen atoms, which may include fused or bridged ring systems, having from three to fifteen carbon atoms, preferably having from three to ten carbon atoms, or preferably having from three to six (C₃-C₆) carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl.

“Cycloalkylalkyl” refers to a radical of the formula —R_{b}R_{c} where R_{b} is an alkylene chain and R_{c} is a cycloalkyl radical as defined above.

“Aryl” means an aromatic carbocyclic moiety such as phenyl or naphthyl (i.e., naphthalenyl) (1-or 2-naphthyl) or anthracenyl (e.g., 2-anthracenyl).

“Arylalkyl” (e.g., phenylalkyl) means an alkyl having at least one alkyl hydrogen atom replaced with an aryl moiety, such as —CH₂-phenyl (i.e., benzyl), —CH═CH-phenyl, —C(CH₃)═CH-phenyl, and the like.

“Heteroaryl” refers to a 5-to 14-membered ring system radical comprising hydrogen atoms, one to thirteen ring carbon atoms, one to six ring heteroatoms selected from the group consisting of nitrogen, oxygen and sulfur, and at least one aromatic ring. For purposes of embodiments of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, dihydroquinolinyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl).

“Heteroarylalkyl” refers to a radical of the formula —R_{b}Rₐ where R_{b} is an alkylene chain and R_{d} is a heteroaryl radical as defined above.

“Halogen” or “halo” means fluoro, chloro, bromo, and iodo.

“Haloalkyl” refers to a halo-substituted alkyl, i.e., alkyl in which at least one hydrogen atom is replaced with halogen. “Perhaloalkyl” refers to haloalkyl in which all of the hydrogens are replaced by halogens. Examples of haloalkyls include trifluomethyl, difluorobromomethyl, difluorochloromethyl, 1,1,2,2,3,3,3-heptafluoropropyl and the like. In certain embodiments, the halo substituents of a haloalkyl or perhaloalkyl may be the same (e.g., all of the halo substituents are fluoro) or different (e.g., the halo substituents may be a mixture of any two or more of fluoro, chloro, bromo or iodo). The alkyl moiety of a haloalkyl may be optionally substituted by one or more substituents as defined herein.

“Haloalkoxy” refers to a substituted alkoxy, means an alkoxy moiety having at least one hydrogen atom replaced with halogen, such as chloromethoxy and the like.

“Heterocycle” refers to a stable 3 to 18 membered ring radical including, as ring atoms, at least one carbon atom and from one to five heteroatoms selected from the group consisting of nitrogen, oxygen and sulfur. For purposes of this disclosure, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; and the nitrogen atom may be optionally quaternized; and the heterocyclyl radical may be partially or fully saturated. Examples of such heterocyclyl radicals include, but are not limited to, dioxolanyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted

All the above groups may be “optionally substituted,” i.e., either substituted or unsubstituted. The term “substituted” as used herein means any of the above groups (i.e., alkyl, alkoxy, alkoxyalkyl, aryl, aralkyl, heterocyclyl, heterocyclylalkyl, heteroaryl, heteroarylalkyl and/or trifluoroalkyl), may be further functionalized wherein at least one hydrogen atom is replaced by a bond to a non-hydrogen atom substituent. Unless stated specifically in the specification, a substituted group may include one or more substituents selected from: oxo, —CO₂H, nitrile, nitro, —CONH₂, hydroxyl, thiooxy, alkyl, alkylene, alkoxy, alkoxyalkyl, alkylcarbonyl, alkyloxycarbonyl, aryl, aralkyl, arylcarbonyl, aryloxycarbonyl, aralkylcarbonyl, aralkyloxycarbonyl, aryloxy, cycloalkyl, cycloalkylalkyl, cycloalkylcarbonyl, cycloalkylalkylcarbonyl, cycloalkyloxycarbonyl, heterocyclyl, heteroaryl, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, thioalkyl triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double-or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced

with —NR_{g}C(═O)NR_{g}Rₕ, —NR_{g}C(═O)ORₕ, —NR_{g}SO₂Rₕ, —OC(—O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —CH₂SO₂R_{g}, —CH₂SO₂NR_{g}Rₕ, —SH, —SR_{g} or —SSR_{g}. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents.

### Use and Method of Treatment

As disclosed herein, various embodiments provide compounds of any one of Formulae (I), (II), (III), (IV) or (V) (or their respective substructures) for preventing or treating a condition, disease, or disorder associated with SLC26A3-mediated anion exchange. In more specific embodiments, the condition, disease, or disorder associated with SLC26A3-mediated anion exchange include chronic idiopathic constipation (CIC), opioid-induced constipation (OIC), constipation-predominant irritable bowel syndrome (IBS-C), CF-associated constipation, meconium ileus, distal intestinal obstruction syndrome, and hyperoxaluria seen in calcium oxalate kidney stone disease, enteric hyperoxaluria and primary hyperoxaluria.

Also provided herein is a method of inhibiting SLC26A3 comprising: contacting (a) a cell that expresses SLC26A3 and (b) a pharmaceutical composition comprising a compound of Formulae (I), (II), (III), (IV) or (V), in an amount effective and under conditions and for a time sufficient to inhibit SLC26A3-mediated anion (Cl⁻, HCO₃⁻, oxalate) exchange.

In a further embodiment, the method further comprises administering, simultaneously or sequentially with the compound of Formulae (I), (II), (III), (IV) or (V) and an NHE3 inhibitor.

In more specific embodiments, NHE3 inhibitor is tenapanor, and the condition or disorder is refractory constipation.

Another embodiment provides a method of decreasing urinary oxalate excretion in a subject in need thereof, the method comprising administering to the subject a therapeutically effective amount of a compound of Formulae (I), (II), (III), (IV) or (V), as described herein.

### EXAMPLES

### Example 1

### Extracellular Action Of SLC26A3 Inhibitors

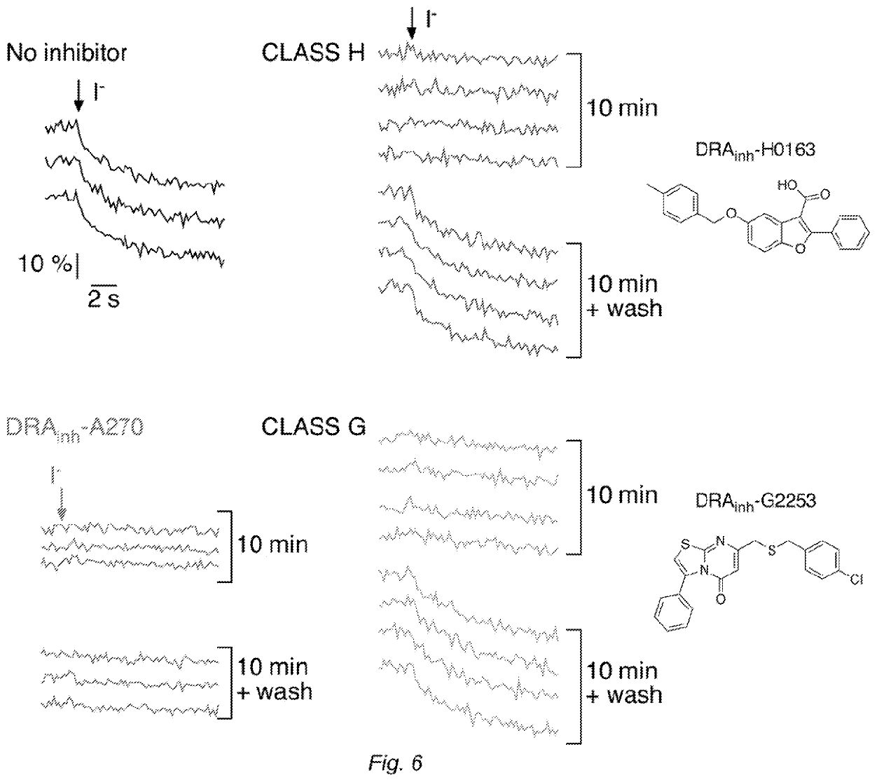

FIG. 6 demonstrates the site of action of two of the SLC26A3 inhibitors of the present disclosure. Cells were incubated for 10 min with indicated compounds at 5 times their IC50 for DRA inhibition. Inhibition was then measured without washout and at 20-30 second after washout. The data show rapid reversal of inhibition for the G and H class compound but no reversal for a known DRA inhibitor (A270), structure shown below:

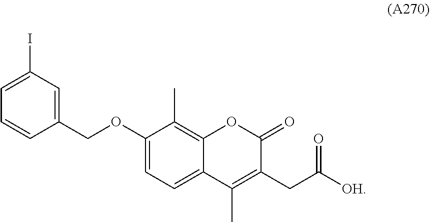 [SMILES: Cc1c(CC(=O)O)c(=O)oc2c(C)c(OCc3cccc(I)c3)ccc12]

This result supports an extracellular site of action for the G and H class compounds, which may be developed into membrane-impermeable, non-absorbable DRA inhibitors.

### Example 2

### SLC26A3 Inhibition Blocks Intestinal Oxalate Absorption

It is demonstrated herein that inhibition of colonic anion exchanger SLC26A3 inhibits oxalate transport at physiological levels of oxalate, making SLC26A3 inhibitors effective therapy for idiopathic hyperoxaluria, enteric hyperoxaluria, primary hyperoxaluria and calcium oxalate nephrolithiasis.

DRAᵢₙₕ-A270 inhibits SLC26A3-mediated oxalate transport in transfected cells

In order to determine the effects of DRAᵢₙₕ-A270 on oxalate transport at physiologically relevant concentrations, transepithelial oxalate transport was measured in Fischer rat thyroid (FRT) monolayers expressing SLC26A3 (FRT-A3 cells) permeable filters and exposed to a 500 μM oxalate concentration gradient.

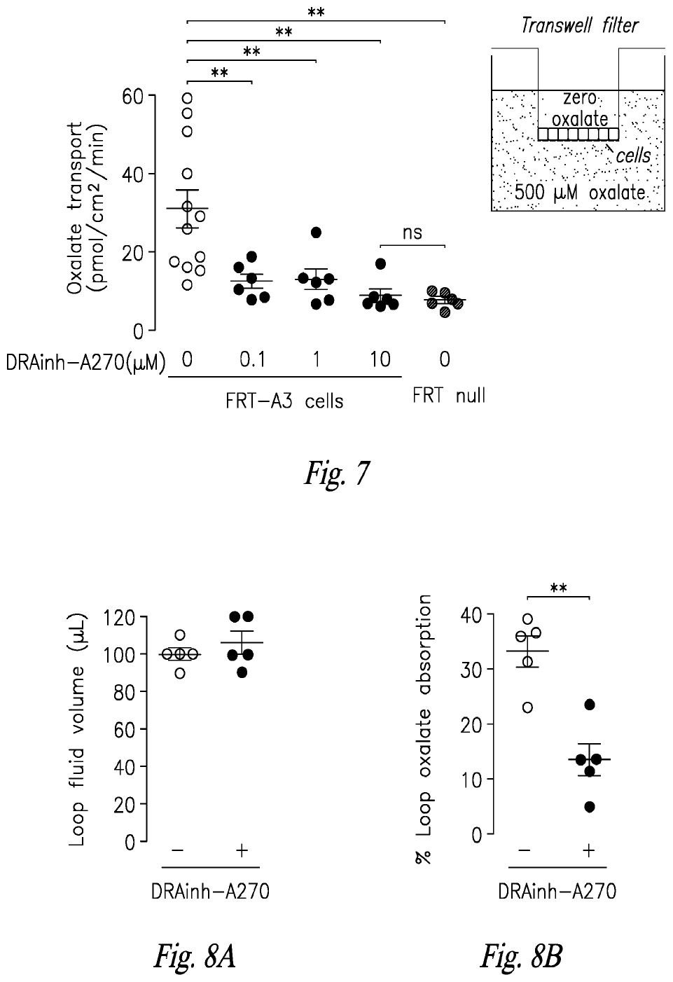

FIG. 7 shows (inset) schematically an oxalate transport assay in FRT cells grown on permeable filters, with 500 μM basolateral to apical oxalate gradient. Oxalate transport rate plotted for studies done in FRT cells expressing SLC26A3 (FRT-A3) pretreated with indicated concentrations of DRAᵢₙₕ-A270 (or DMSO control) for 15 min prior to application of the oxalate gradient. FRT cells not expressing SLC26A3 (FRT-null) are shown as controls. Mean±S.E.M., n=6-12 wells per condition. One-way analysis of variance with post hoc Newman-Keuls multiple comparisons test, **p<0.01, ns: not significant.

As shown, FRT-A3 cells had ˜4.5 fold increased oxalate transport compared to FRT-null cells, and the increased oxalate transport in FRT-A3 cells was largely blocked by DRAᵢₙₕ-A270 pretreatment even at 0.1 μM.

### DRAᵢₙₕ-A270 Prevents Intestinal Oxalate Absorption

To investigate DRAᵢₙₕ-A270 inhibition of intestinal oxalate transport, experiments were conducted using closed distal colonic loops in which SLC26A3 is the major apical membrane anion transporter. Colonic loops were injected with the solution containing 500 μM sodium oxalate and loop fluid was removed at 60 min to quantify remaining oxalate concentration. To avoid confounding effects of fluid absorption, Cl⁻-free solution with amiloride (to block ENaC) was used.

FIG. 8A shows loop fluid volume at 60 min in mouse distal colonic loops injected at 0 min with 100 μL of Cl free HEPES-buffered saline containing 500 μM sodium oxalate (+20 μM amiloride) with and without 10 μM DRAᵢₙₕ-A270. N=5 loops per group.

FIG. 8B shows the percentage absorption of luminal oxalate (500 μM, at 0 min) in mouse distal colonic loops at 60 min in the presence and absence of 10 μM DRAᵢₙₕ-A270.

As shown, luminal DRAᵢₙₕ-A270 reduced oxalate absorption by ˜70% compared to vehicle control in this model.

The various embodiments described above can be combined to provide further embodiments. All U.S. patents, U.S. patent application publications, U.S. patent applications, foreign patents, foreign patent applications, and non-patent publications referred to in this specification are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 63/227,926 filed Jul. 30, 2021, the entirety of which is incorporated by reference herein.

1.-7. (canceled)

8. A method for preventing or treating a condition, disease, or disorder associated with SLC26A3-mediated anion exchange in a subject in need thereof, the method comprising administering to the subject a therapeutically effective amount of a compound of formula (I), (II), (III), (IV) or (V):
wherein,
formula (I) is represented by the following structure: 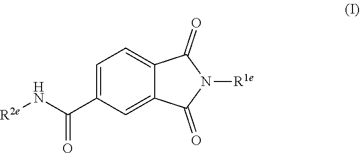
wherein,
R^{1e} is (i) phenyl optionally substituted with one or more substituents selected from the group consisting of C₁₋₆alkyl, halo, —NO₂, phenyl, and C₁₋₆alkoxy; (ii) C₃₋₆alkyl, or (iii) heterocyclylC₁₋₄alkyl; and
R^{2e} is carboxy-substituted phenyl, optionally further substituted with one or more substituents selected from the group consisting of C₁₋₆alkyl, halo, hydroxyl and C₁₋₆alkoxy;
formula (II) is represented by the following structure: 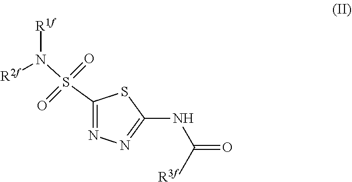
wherein,
R^{1f} is hydrogen or C₁₋₃alkyl;
R^{2f} is phenyl optionally substituted with one or more substituents selected from the group consisting of C₁₋₆alkyl, halo, and C₁₋₆alkoxy; or
R^{1f} and R^{2f} together with the nitrogen to which they are connected form a heteroaryl optionally substituted with C₁₋₃alkyl; and
R^{3f} is
(i) phenyl optionally substituted with one or more substituents selected from the group consisting of C₁₋₆alkyl, halo, —NO₂, and C₁₋₆alkoxy;
(ii) C₅₋₆ cycloalkyl or C₅₋₆ heteroaryl;
(iii) 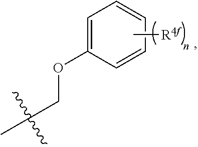
wherein n is 0, 1 or 2, R^{4f} is C₁₋₆alkyl, halo, or C₁₋₆alkoxy.
formula (III) is represented by the following structure: 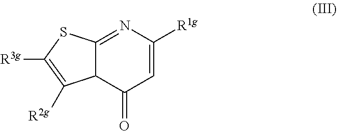
wherein,
R1g is 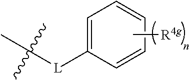
wherein, n is 0, 1, 2, or 3;
L is —(CH₂)ₘ—X—(CH₂)ₚ—, wherein m and p are independently 0 or 1, X is —O—, —S—, —N(R^{5g})—, or —OC(O)—;
R^{2g} is phenyl optionally substituted with one or more substituents selected from the group consisting of C₁₋₄alkyl and C₁₋₄alkoxy;
R^{3g} is hydrogen or C₁₋₄alkyl;
R^{4g} is C₁₋₄alkyl, halo, or C₁₋₄alkoxy; or two adjacent R^{4g} and the carbons to which they are attached form a 5-member or 6-member heteroaryl; and
R^{5g} is hydrogen or C₁₋₃alkyl;
formula (IV) is represented by the following structure: 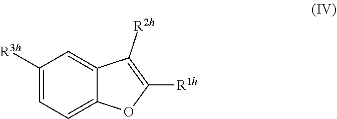
wherein,
R^{1h} is C₁₋₃alkyl or phenyl optionally substituted with one or more substituents selected from the group consisting of C₁₋₄alkyl, halo, and C₁₋₄alkoxy;
R^{2h} is —COOR^{5h};
R^{3h} is 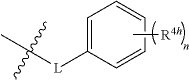
wherein, n is 0, 1, or 2;
L is —X—(CH₂)ₘ—Y—(CH₂)ₚ—, wherein m and p are independently 0 or 1, X is —O— or —S—, Y is a direct bond or —C(O)O—;
R^{4h} is C₁₋₄alkyl or halo; and
R^{5h} is hydrogen or C₁₋₄alkyl; and
formula (V) is represented by the following structure: 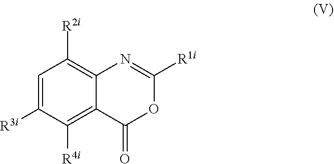
wherein,
R¹ⁱ is
(i) phenyl optionally substituted with one or more substituents selected from the group consisting of C₁₋₄alkyl, halo, —NO₂, C₁₋₄alkoxy and —NHC(O)—R⁵ⁱ; or
(ii) 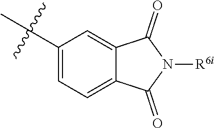
R²ⁱ is hydrogen or C₁₋₃alkyl;
R³ⁱ and R⁴ⁱ are hydrogen; and
R⁵ⁱ is
(i) C₁₋₄alkyl;
(ii) -L-phenyl, wherein phenyl is optionally substituted with one or more substituents selected from the group consisting of C₁₋₄alkyl, halo, C₁₋₄alkoxy and C(O)O—C₁₋₄alkyl; L is a direct bond or —(CH₂)ₘ—X—, and X is O or S;
R⁶ⁱ is C₁₋₄alkyl or phenyl optionally substituted with C₁₋₄alkyl.

9. The method of claim 8, wherein the compound of Formula (I) is represented by Formula (Ia): 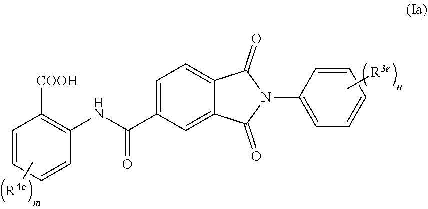
wherein,
m is 0, 1 or 2;
n is 1, 2 or 3;
R^{3e} is C₁₋₆alkyl, halo, —NO₂, phenyl, and C₁₋₆alkoxy;
R^{4e} is C₁₋₆alkyl, halo, hydroxyl or C₁₋₆alkoxy.

10. The method of claim 8, wherein the compound of Formula (I) has one of the following structures:

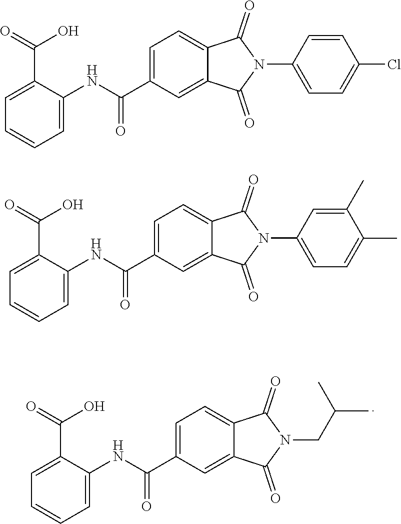 [SMILES: O=C(Nc1ccccc1C(=O)O)c1ccc2c(c1)C(=O)N(c1ccc(Cl)cc1)C2=O ; Cc1ccc(N2C(=O)c3ccc(C(=O)Nc4ccccc4C(=O)O)cc3C2=O)cc1C ; CC(C)CN1C(=O)c2ccc(C(=O)Nc3ccccc3C(=O)O)cc2C1=O]

11. The method of claim 8, wherein the compound of Formula (II) has one of the following structures:

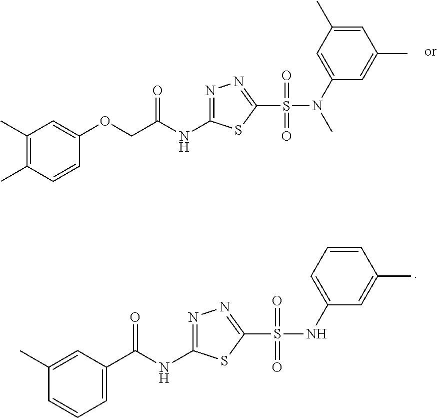

12. The method of claim 8, wherein the compound of Formula (III) has one of the following structures:

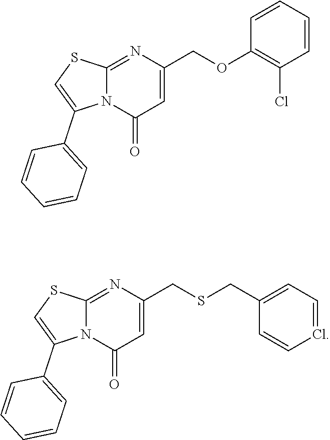

13. The method of claim 8, wherein the compound has one of the following structures:

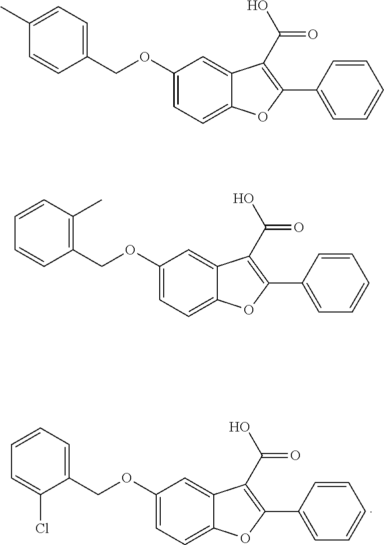 [SMILES: Cc1ccc(COc2ccc3oc(-c4ccccc4)c(C(=O)O)c3c2)cc1 ; Cc1ccccc1COc1ccc2oc(-c3ccccc3)c(C(=O)O)c2c1 ; O=C(O)c1c(-c2ccccc2)oc2ccc(OCc3ccccc3Cl)cc12]

14. The method of claim 8, wherein the condition, disease, or disorder associated with SLC26A3-mediated anion exchange is constipation or hyperoxaluria.

15. The method of claim 8, wherein the condition, disease, or disorder associated with SLC26A3-mediated anion exchange is constipation or hyperoxaluria.

16. The method of claim 8, wherein the condition, disease, or disorder associated with SLC26A3-mediated anion exchange is constipation or hyperoxaluria.

17. The method of claim 8, wherein the condition, disease, or disorder associated with SLC26A3-mediated anion exchange is chronic idiopathic constipation (CIC), opioid-induced constipation (OIC), constipation-predominant irritable bowel syndrome (IBS-C), CF-associated constipation, meconium ileus, distal intestinal obstruction syndrome, calcium oxalate kidney stone disease, enteric hyperoxaluria, or primary hyperoxaluria.
